(19) United States
(12) Patent Application Publication
(10) Pub. No.: US 2012/0310084 A1
(43) Pub. Date: Dec. 6, 2012

(54) MEDICAL FLUID INJECTION DEVICE AND FLUOROSCOPIC IMAGING SYSTEM

(75) Inventor: Shigeru Nemoto, Tokyo (JP)

(73) Assignee: NEMOTO KYORINDO CO., LTD., Tokyo (JP)

(21) Appl. No.: 13/577,034

(22) PCT Filed: Feb. 2, 2011
(86) PCT No.: PCT/JP2011/052120

§ 371 Date: Aug. 3, 2012

(30) Foreign Application Priority Data
Feb. 5, 2010 (JP) 2010-024037

(51) Int. Cl. A61B 6/00

(52) U.S. Cl. 600/431

(57) ABSTRACT

The medical fluid injector (100) includes injector head (100) and controller (150). The controller (150) performs processing of receiving an input of a distance from a first position to a second position of a patient, processing of receiving an input of time t1 for the contrast medium to reach the first position, and processing of receiving an input of time t2 for the contrast medium to reach the second position. The controller further calculates a flow rate of the contrast medium based on the distance from the first position to the second position and times t1 and t2, determines a moving speed of a bed of the fluoroscopic imaging apparatus in association with the flow rate, and displays the moving speed of the bed on a display of the controller.

### TECHNICAL FIELD

The present invention relates to a medical fluid injector and a fluoroscopic imaging system. Specifically, the present invention relates to a medical fluid injector and a fluoroscopic imaging system capable of properly and successfully imaging an object such as arteries from abdomen to leg parts with a smaller amount of contrast medium than conventional means even in imaging over a wide range.

### BACKGROUND ART

Currently employed medical imaging diagnosis apparatuses include CT (Computed Tomography) scanners, MRI (Magnetic Resonance Imaging) apparatuses, PET (Positron Emission Tomography) apparatuses and the like. In using the abovementioned imaging apparatuses, a contrast medium or physiological saline (hereinafter also referred to simply as a medical fluid) is often injected into the patient's body. Various injectors (injection heads) for automatically performing the injection have conventionally been proposed.

With respect to an imaging with such a contrast medium, for example the Patent Document 1 discloses an injector wherein firstly a contrast medium is injected into a patient (test shot), while a scanner continuously applies a small dose of radiation. This achieves a monitoring scan in which the X-ray image of a desired position is obtained in real time. Then, an operator checks the timing when the contrast medium is properly distributed in a certain region based on images obtained by the monitoring scan. The operator then operates the CT scanner apparatus to start a main scan based on that timing.

Patent Document 1: Japanese Patent Laid-Open No. 2000-325341

### DISCLOSURE OF THE INVENTION

### Problems to be Solved by the Invention

When imaging for example arteries from abdomen to leg parts, it is necessary to inject a large amount of contrast medium so as to properly visualize the overall blood vessel. From the viewpoint of side effects, cost and the like of contrast medium, there is a need to develop an apparatus capable of performing favorable visualization with a smaller amount of contrast medium than conventional means. Especially, for a patient having a high creatinine level or a patient readily suffering from side effects, the problem of the side effects is serious.

The present invention has been made in view of the abovementioned problem, and it is an object thereof to provide a medical fluid injector and a fluoroscopic imaging system capable of properly imaging an object such as arteries from abdomen to leg parts with a smaller amount of contrast medium than conventional even in imaging over a wide range.

### Means for Solving the Problems

To solve the abovementioned problem, the present invention provides a medical fluid injector including an injector head injecting a contrast medium into a patient and a controller for setting an injection condition for the contrast medium, the controller being configured to communicate with a fluoroscopic imaging apparatus,

wherein the controller performs:

processing of receiving an input of a distance from a first position to a second position of a patient to be imaged by the fluoroscopic imaging apparatus;

processing of receiving an input of a time t1 for the contrast medium to reach the first position;

processing of receiving an input of a time t2 for the contrast medium to reach the second position;

processing of calculating a flow rate of the contrast medium based on the distance from the first position to the second position and the times t1 and t2;

processing of determining a moving speed of a bed of the fluoroscopic imaging apparatus in association with the flow rate; and

processing of displaying the moving speed of the bed on a display of the controller.

“Determining the moving speed of the bed of the fluoroscopic imaging apparatus in association with the flow rate” intends to mean that a flow rate may be identical to the bed-moving speed or may be slightly different from the bed-moving speed.

In the “processing of receiving the input of the time t1” and the “processing of receiving the input of the time t2,” the times t1 and t2 may be input manually by a doctor or the like to the controller. Or, the times t1 and t2 may be measured by the fluoroscopic imaging apparatus and data relating to the times may be automatically input to the controller via a communication between the fluoroscopic imaging apparatus and the controller.

According to such a configuration, the bed of the fluoroscopic imaging apparatus can be moved in accordance with flow rate of the contrast medium to perform the scan. Accordingly, for example, even when a wide imaging range including blood vessels from abdomen to leg parts is to be imaged, the visualization can be properly achieved with a small amount of contrast medium. In addition, according to the present imaging method, the exposure can be further reduced than conventional means.

According to another aspect of the present invention, the controller further performs processing of transmitting information relating to the bed-moving speed to the fluoroscopic imaging apparatus.

The controller further performs:

processing of determining a scan-start time by adding a predetermined additional time to the time t1; and

processing of transmitting data relating to said scan-start time to the fluoroscopic imaging apparatus.

According to this configuration, since the data is automatically transmitted from the controller to the fluoroscopic imaging apparatus, the fluoroscopic imaging apparatus can automatically set the scan condition based on this data, to eliminate the need for setting of the condition in the fluoroscopic imaging apparatus by a doctor or the like.

According to another aspect of the present invention, the controller further performs processing of receiving an input of a weight of a patient, and in the processing of determining the scan-start time, the controller refers to a correspondence table representing a relation between a segment of the weight of the patient and the additional time to determine the additional time corresponding to the input weight, and adds the additional time to the time t1 to determine the scan-start time appropriate for the patient.

The “controller” may be provided as an independent unit or may be provided as part of functions of another device. For example, the controller may be incorporated in part of a control device of the fluoroscopic imaging apparatus.

### Advantage of the Invention

As described above, according to the present invention, the medical fluid injector and the fluoroscopic imaging system can be provided which are capable of properly and successfully imaging an object such as arteries from abdomen to leg parts with a smaller amount of contrast medium than conventional means even in imaging over a wide range.

### BRIEF DESCRIPTION OF THE DRAWINGS

FIG. 1 is a perspective view of a medical fluid injector and a fluoroscopic imaging system including the same according to an embodiment of the present invention.

FIG. 2 is a diagram showing an image displayed on a display of a controller.

FIG. 3 is a flow chart showing a series of operations in the system in FIG. 1.

FIG. 4 is a graph schematically showing the relationship between patient's weights and peak times of TDCs.

### BEST MODE FOR CARRYING OUT THE INVENTION

An embodiment of the present invention will hereinafter be described with reference to the drawings.

As shown in FIG. 1, a fluoroscopic imaging system 1 according to the present embodiment includes a medical fluid injector 100 and a fluoroscopic imaging apparatus 300 capable of operating in association with the injector. The injector 100 includes an injector head 110 for injecting a contrast medium into a patient and a controller 150 for setting condition(s) for injection.

The injector head 110 may be of a dual-type head on which a syringe filled with the contrast medium and a syringe filled with physiological saline are configured to be mounted, or may be of a single-type head on which one syringe filled with the contrast medium is configured to be mounted. The syringe may be a conventionally known pre-filled type syringe in which a piston member is slidably inserted into a tubular cylinder member.

The injector head 110, although not shown, has piston-driving mechanisms for pushing the piston member of each syringe. The piston-driving mechanism may be provided with a load cell for detecting a pressure with which the piston member is pushed. The injector head 110 may also have a tag reader (not shown) for reading data of an IC tag attached to the syringe.

The IC tag may store data relating to a syringe (including identification data of syringe such as a lot number, pressure limit of cylinder member, inner diameter of cylinder member, and stroke of piston member), or data relating to medical fluid filled in the syringe (including name (product name), component information including iodine amount, viscosity, or the expiration date).

The controller 150 has a display 151 of a touch panel type. For example, an image for setting injection conditions is displayed on display 151 (described later in detail). Information relating to the medical fluid in the syringe, injection pressure of the medical fluid during injection (force to push the piston member) and the like may be displayed on the display 151. A plurality of displays may be provided, or a sub-display may be provided for the injector head.

As shown in FIG. 1, the controller 150 is connected to both the injector head 110 and the fluoroscopic imaging apparatus 300 (described later in detail) and can communicate with them. While FIG. 1 shows an example of wired connection, it goes without saying that wireless connection may be used for the communication between them.

The fluoroscopic imaging apparatus 300 can be realized by using a known apparatus. The apparatus 300 includes a bed 303 movable horizontally with a patient lying thereon, an apparatus body 302 for shooting an image of a patient during the movement of bed 303 at a predetermined speed, and a control section (not shown) having a monitor, a keyboard and the like. In fluoroscopic imaging apparatus 300 shown in FIG. 1, a patient lies on bed 303 with his head toward apparatus body 302 and is imaged from head toward leg by apparatus body 302. The imaging may also be performed from leg toward head.

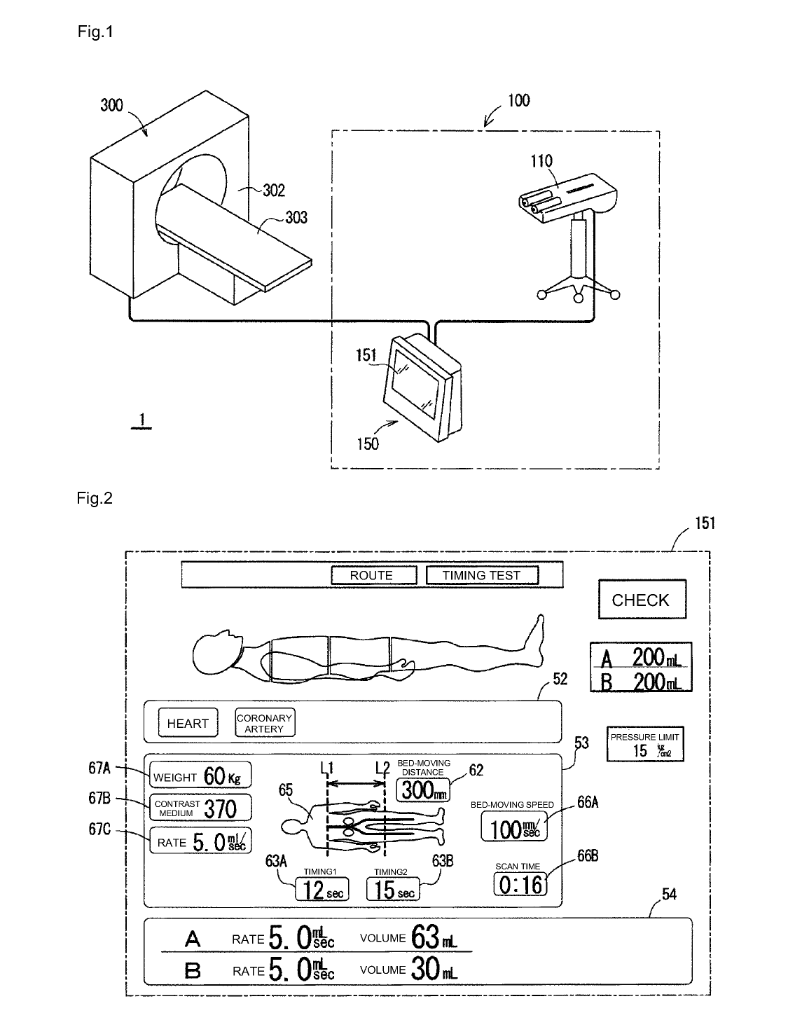

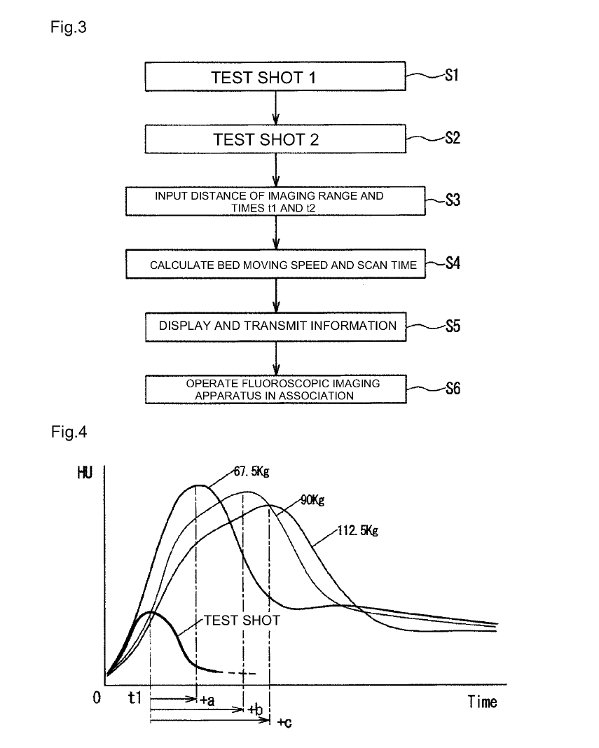

FIG. 2 shows an image displayed on display 151 of the medical fluid injector of the present embodiment. As shown in FIG. 2, a plurality of windows 52 to 54 are displayed on display 151. In the window 53, a schematic image 65 representing a human body viewed from above is displayed. The human body schematic image 65 includes schematically shown arteries extending from abdomen to leg parts.

Two vertical lines L1 and L2 cross the human body schematic image 65. The position of line L1 corresponds to a beginning point of an imaging range, whereas the position of line L2 corresponds to an end point of the imaging range.

An image button 63A is displayed below the line L1, the button 63A is used for inputting time t1 that represents a time when the contrast medium will reach the position L1 (first position). Similarly, an image button 63B is displayed below line L2, the button 63B is used for inputting time t2 that represents a time when the contrast medium will reach the position L2 (second position).

A doctor can measure the times t1 and t2 during a monitoring scan performed prior to a main scan. Alternatively, times t1 and t2 may be automatically measured by the fluoroscopic imaging apparatus in the monitoring scan.

An image button 62 for inputting a distance between lines L1 and L2 (corresponding to the bed-moving distance) is displayed above human body schematic image 65. Three image buttons 67A to 67C are displayed vertically in line on the left of human body schematic image 65. The image button 67A is provided for inputting weight of a patient, the image button 67B is provided for inputting a concentration of contrast medium, and the image button 67C is provided for inputting an injection rate of medical fluid.

A display section 66A for a bed-moving speed (described later in detail) calculated by controller 150 and a display section 66B for a scan-start time are displayed on the right of the human body schematic image 65.

More specifically, the controller 151 performs the following processing including:

processing of receiving an input of the distance from the first position to the second position of the patient to be imaged by fluoroscopic imaging apparatus 300;

processing of receiving an input of time t1 for the contrast medium to reach the first position;

processing of receiving an input of time t2 for the contrast medium to reach the second position;

processing of calculating a flow rate of the contrast medium based on the distance from the first position to the second position and times t1 and t2;

processing of determining the moving speed of bed 303 of the fluoroscopic imaging apparatus in association with the flow rate;

processing of transmitting the information about the bed-moving speed to fluoroscopic imaging apparatus 300;

processing of determining the scan-start time by adding a predetermined additional time to time t1; and

processing of transmitting the information about the scan-start time to fluoroscopic imaging apparatus 300.

The fluoroscopic imaging apparatus 300 in FIG. 1 is configured to communicate with the controller 150. As described later, the apparatus moves the bed 303 in accordance with information relating to the bed-moving speed transmitted from the controller 150. The fluoroscopic imaging apparatus 300 receives the information relating to the scan time from the controller 150. The apparatus will start imaging (main scan) at a timing when the scan time has elapsed since the start of the medical fluid injection.

Next, description is made of the operation of fluoroscopic imaging system 1 of the present embodiment configured as described above.

A first test shot is conducted firstly, to measure the time t1 for the contrast medium to reach the first position (step S1). In this test shot, a small amount of contrast medium (for example 10 ml) is injected into a patient from injector head 110 so that a TDC (Time Density Curve) can be obtained. The peak time of the curve will be set to the time t1.

Next, a second test shot is conducted to measure time t2 for the contrast medium to reach the second position (step S2). This step can be performed similarly to the abovementioned step. The peak time of the resulting TDC curve will be set to the time t2.

The times t1 and t2 thus can be obtained through the two test shots.

It should be noted that a bolus tracking method might be used to measure times t1 and t2, instead of the test injection mentioned above. In the bolus tracking method, a time for contrast medium to reach predetermined position is measured while monitoring a section view of the ROI (Region Of Interest) in real time.

Next, times t1 and t2 measured in the above steps and the distance of the imaging range are input through the display 151 of the controller 150. Specifically, the operator touches the image button 62 shown in FIG. 2 and inputs a numeric value with a ten-key numeric keypad displayed in response to the touch (by way of example), not shown, to input a distance of the imaging range. In this example, the distance of the imaging range is 300 mm.

The time t1 may be input by an operator for example by touching the image button 63A and selecting one of a plurality of options displayed as a pull-down menu (by way of example) in response to the touch (in this example, 12 sec). Similarly, the time t2 may be input by the operator by touching the image button 63B and selecting one of a plurality of options displayed as a pull-down menu (by way of example) in response to the touch (in this example, 15 sec).

The weight of the patient and the iodine amount of the contrast medium are input through display 151. Specifically, the operator touches the image button 67A and inputs a numeric value with a ten-key numeric keypad (by way of example), not shown, displayed in response to the touch, to set the patient's weight. The operator also touches the image button 67B and selects one of a plurality of options displayed as a pull-down menu (by way of example) in response to the touch, to set the iodine amount.

The image 67C represents a rate of medical fluid injection; 5.0 mL/sec is displayed in this example. Although not limited particularly, an operator may touch the image button 67C to input a numeric value with a ten-key numeric keypad (by way of example), not shown, displayed in response to the touch, to adjust the value.

After the input of both the distance of the imaging range and times t1 and t2, the controller 150 calculates a flow rate of the contrast medium based on these data (step S4). Specifically, in this example, the time t1 is 12 sec, the time t2 is 15 sec, and the distance of imaging range is 300 mm, the flow rate of the contrast medium is calculated to 10 mm/sec. The computer apparatus 150 then determines the flow rate 100 mm/sec as a bed-moving speed. The apparatus displays this speed in the display section 66A of the display.

Although in the above description the bed-moving speed is determined by using a flow rate 100 mm/sec of the contrast medium (that is, the flow rate of the contrast medium is identical to the bed-moving speed), the present invention is not limited thereto.

The computer apparatus 150 also calculates a timing (scan-start time) at which the fluoroscopic imaging apparatus 300 starts a scan based on time t1 for the contrast medium to reach the first position. Specifically, the computer apparatus 150 adds a predetermined additional time (4 seconds in this example) to time t1 (12 seconds), and determines the resulting “16 seconds” as the scan-start time. The controller 150 then displays the scan-start time in the display section 66B of the display.

The controller 150 may have a correspondence table (not shown) representing a correspondence between segments of the patient's weight and additional times. The controller determines an additional time with reference to the correspondence table. For example, when a patient's weight is equal to or less than 67.5 Kg, additional time is +4 sec (see “a” in FIG. 4). When a patient's weight is equal to or less than 90.0 Kg, additional time is +7 sec (see “b”). When a patient's weight is equal to or less than 112.5 Kg, additional time is +11 sec (see “c”).

The additional time may be determined with reference to the correspondence table, or may be calculated by substituting a patient's weight into a predetermined calculation expression.

The reason why the additional time becomes longer as the patient's weight is heavier is based on the following theory: As the patient's weight is heavier, the injection amount of contrast medium is increased, and the peak time of the TDC (Time Density Curve) shifts as shown in FIG. 4. Therefore, it is necessary to delay the starting timing of the fluoroscopic imaging apparatus in accordance with the shift.

To take it into account in the present embodiment the predetermined additional time for example, 4, 7, or 11 sec determined in view of patient's weight is added to time t1 (12 sec) in the test shot. This enables the apparatus to start scanning at the appropriate timing in view of patient's weight.

The description of the operation of the present system is now continued. After the bed-moving speed and the scan-start time are determined at the abovementioned step (S4), the controller 150 transmits the information to the fluoroscopic imaging apparatus 300 (step S5). The preparation prior to the main scan is completed by the steps so far performed.

The fluoroscopic imaging apparatus 300 then moves the bed 303 at the bed-moving speed (100 mm/sec in this example) transmitted from the controller 150. After the elapse of the predetermined time since the injection start by the injector head 110 (after 16 sec in this example), the fluoroscopic imaging apparatus 300 controls the apparatus body 302 to start scanning while moving the bed (step S6). The scan will be conducted from the first position to the second position of the patient.

According to the system 1 of the present embodiment as described above, the scan is performed while the bed of the fluoroscopic imaging apparatus is moved in accordance with the speed at which the contrast medium flows. Thus, for example in imaging the arteries from abdomen to leg parts, favorable visualization can be performed with a small amount of contrast medium.

Time for a contrast medium to reach an abdomen part or a leg part can vary according to individuals. However, in the system of the present embodiment, the bed-moving speed is calculated by using times t1 and t2 measured in the test shot before the main scan, therefore appropriate bed-moving speed for each patient can be obtained. One of the reasons why the time contrast medium to reach an abdomen part or a leg part can vary is that the bloodstream speed varies among patients due to differences in body weight, height, blood vessel conditions (for example, hardness of blood vessels) and the like.

According to the present embodiment, the need of the work by a doctor or the like to set conditions through the fluoroscopic imaging apparatus 300 can be eliminated. Because, in the embodiment described above, data relating to “bed-moving speed” and “scan-start time” determined by the controller 150 is configured to be transmitted to the fluoroscopic imaging apparatus 300, and the apparatus 300 can automatically operate in association therewith (that is, the imaging apparatus 300 will operate based on the scan-start time).

The present invention is not limited to the above. The system can be configured so that the display 151 just displays information of the “bed-moving speed” and “scan-start time”. Even in such a configuration, the advantages of the present invention described above can be achieved by a doctor or the like checking the information and setting the condition for the fluoroscopic imaging apparatus through manual input.

In the system of the present embodiment, the scan is performed while the bed of the fluoroscopic imaging apparatus is moved in accordance with the speed at which the contrast medium flows. Thus, exposure for a patient can be reduced as compared with the conventional system. Especially, the imaging apparatus recently has improved performance and shortened imaging time. The effect of reducing the exposure described above can be achieved more significantly, if such an imaging apparatus is used.

The embodiment described above has shown the example in which times t1 and t2, and the distance between L1 and L2 are input manually by the doctor or the like. However, the present invention is not limited thereto. For example, the fluoroscopic imaging apparatus 300 may communicate with the controller 150 so that the apparatus 300 can automatically input data for times t1 and t2 and/or distance between L1 and L2 to the controller. Alternatively, an apparatus can be adopted, which automatically calculate a distance between L1 and L2 when the position of L1 and the position of L2 are input, rather than the apparatus where distance between L1 and L2 is input.

In addition to the above embodiment, input means such as a mouse and a keyboard, rather than the touch panel, may be used to input various information to the controller 150. The controller 150 may be connected to a network such that data including medical fluid injection conditions and scan conditions is supplied to controller 150 over the network.

### DESCRIPTION OF REFERENCE NUMERALS

1 FLUOROSCOPIC IMAGING SYSTEM

52, 53, 54 WINDOW

62, 63A, 63B IMAGE BUTTON

110 INJECTION HEAD

150 CONTROLLER

151 DISPLAY

300 IMAGING DIAGNOSTIC APPARATUS

302 APPARATUS BODY

303 BED

1. A medical fluid injector comprising an injector head for injecting a contrast medium into a patient and a controller for setting an injection condition for the contrast medium, the controller being configured to communicate with a fluoroscopic imaging apparatus,
wherein the controller performs the following processes:
receiving an input of a distance from a first position to a second position of a patient to be imaged by the fluoroscopic imaging apparatus;
receiving an input of a time t1 for the contrast medium to reach the first position;
receiving an input of a time t2 for the contrast medium to reach the second position;
calculating a flow rate of the contrast medium based on the distance from the first position to the second position and the times t1 and t2;
determining a moving speed of a bed of the fluoroscopic imaging apparatus in association with the flow rate; and
displaying the moving speed of the bed on a display of the controller.

2. The medical fluid injector according to claim 1, wherein the controller further performs a process of transmitting information relating to the bed-moving speed to the fluoroscopic imaging apparatus.

3. The medical fluid injector according to claim 1, wherein the controller further performs the following processes:
determining a scan-start time by adding a predetermined additional time to the time t1; and
transmitting data relating to said scan-start time to the fluoroscopic imaging apparatus.

4. The medical fluid injector according to claim 3, wherein the controller further performs a process of receiving an input of a weight of the patient, and in the process of determining the scan-start time, the controller refers to a correspondence table representing a relation between a segment of the weight of the patient and the additional time to determine the additional time corresponding to the input weight, and adds the additional time to the time t1 to determine the scan-start time appropriate for the patient.

5. The medical fluid injector according to any one of claim, wherein the display of the controller being is a touch panel type, wherein the input of the distance from the first position to the second position of the patient;
the input of the time t1 for the contrast medium to reach the first position; and
the input of the time t2 for the contrast medium to reach the second position;
are performed with an automatic input through the communication between the fluoroscopic imaging apparatus and the controller, or performed with manual input by an operator through the touch panel.

6. A fluoroscopic imaging system comprising:
the medical fluid injector according to claim 1; and
a fluoroscopic imaging apparatus including an apparatus body for imaging a desired position of a patient injected with a contrast medium and a bed.

7. The fluoroscopic imaging system according to claim 6, wherein the fluoroscopic imaging apparatus is configured to communicate with the medical fluid injector.

8. The fluoroscopic imaging system according to claim 7, wherein the medical fluid injector is the medical fluid injector according to claim 2, and the fluoroscopic imaging apparatus receives the information relating to the bed-moving speed from the controller, and moves the bed at said moving speed.

9. The fluoroscopic imaging system according to claim 7, wherein the medical fluid injector is the medical fluid injector according to claim 3, and the fluoroscopic imaging apparatus receives the information relating to the scan-start time from the controller, and starts imaging when the scan-start time has elapsed after start of injection of the medical fluid.
